(19) United States
(12) Patent Application Publication
(10) Pub. No.: US 2008/0261310 A1
(43) Pub. Date: Oct. 23, 2008

(54) IN VITRO ROOTING OF HOODIA PLANTS

(75) Inventors: Blessed N. OKOLE, Kwazulu Natal (ZA); Nadine MOODLEY, Phoenix (ZA)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO, INC., D/B/A/ UNILEVER, Englewood Cliffs, NJ (US)

(21) Appl. No.: 11/737,911

(22) Filed: Apr. 20, 2007

(51) Int. Cl. C12N 5/04

(52) U.S. Cl. 435/420

(57) ABSTRACT

An in vitro tissue culture process of rooting Hoodia explants by using an inventive combination of phytohormones.

### FIELD OF THE INVENTION

The present invention relates to micropropagation via tissue culture for Hoodia plants, particularly the rooting stage of the micropropagation.

### BACKGROUND OF THE INVENTION

Hoodia genus of plants are succulent desert plants which belong to Apocynaceae family. The Apocynaceae family includes numerous other genera of plants. Hoodia plants grow predominantly in South Africa. Hoodia gordonii also grows in Botswana and Namibia. Certain actives obtainable from Hoodia plants, e.g. steroidal glycosides, have been shown to have appetite suppressant activity and to be useful in weight management products. Many of these species, e.g. Hoodia gordonii, are on the endangered list, so that collection of the wild plants is not possible. Commercial cultivation and harvesting of Hoodia plants has become of interest.

Typically, plants are reproduced by collecting and then planting the seeds. Growing Hoodia from seeds, however, takes a longer period from planting to harvesting and plants that are propagated from seeds have a higher probability of early wilting and death, for instance caused by some seed and soil borne pathogens belonging to the genus (Rhizoctonia, Pythium, Phytophthora and Fusarium species). Propagation via tissue culture method allows one to start with clean, disease free materials, with a high vigor and health. Tissue culture method also achieves a shorter time from planting to harvest. Furthermore, the tissue culture method results in a more exact “cloning” of the genetic profile of a parent plant, allowing cultivation and bulking of an elite line of plants with genetic conformity, over a relatively short period of time.

WO 2006/051334 discloses cell cultures of Hoodia. Cell culture differs from tissue culture. Cell cultures seek not to propagate the plant, but to produce the eventual active (e.g., steroidal glycosides from Hoodia) directly from cultured cells, thus obviating the need for growing the plant. Tissue culture techniques for some plants are disclosed, for example, in US2004/091780 (Parvatam et al.), WO 2005/122752, U.S. Pat. No. 6,815,205, and WO 89/10958.

Unfortunately, identifying the various tissue culture conditions which result in successful propagation and rooting, can be extremely difficult and is mostly an empirical process. Although general tissue culture techniques have been known since 1950s, there is considerable difficulty in predicting the effects of plant growth regulators: this is because of the great differences in culture response between species, cultivars, and even plants of the same cultivar grown under different conditions. “Each type of plant grown requires specific culture conditions the development of which involves a great deal of time and effort.” See WO 2005/122752, p. 1, lines 19-23. The part of the plant that efficiently responds to culture conditions and the preparation of that part, the response to tissue culture medium, the type and amount of plant growth regulators, and growth conditions, and then the conditions for rooting and hardening of the plant, each of these differ enormously from plant species to species and variety to variety. “Thus, inventing conditions for efficient regeneration of plants requires developing specialized knowledge about a given plant.” See Parvatam et al., US 2004/091780, paragraphs 6-9.

Thus, the need remains for an effective tissue culture process for Hoodia plants.

### SUMMARY OF THE INVENTION

The inventive process is an effective, commercially suitable alternative to growing Hoodia plants from seeds. The inventive process is based, in part, on the discovery that the type and relative amounts of auxins and other ingredients are critical in order to attain rooting of Hoodia explants, within at least 2 weeks to 5 months, preferably 3 weeks to 3 months, most preferably 4 weeks to 2 months. Callus formation is preferably to be avoided or minimized, in order to have well developed roots that can function immediately after hardening.

### DETAILED DESCRIPTION OF THE INVENTION

Except in the operating and comparative examples, or where otherwise explicitly indicated, all numbers in this description indicating amounts of material or conditions of reaction, physical properties of materials and/or use are to be understood as modified by the word “about.”

It should be noted that in specifying any range, any particular upper limit can be associated with any particular lower limit.

For the avoidance of doubt the word “comprising” is intended to mean “including” but not necessarily “consisting of” or “composed of.” In other words, the listed steps or options need not be exhaustive.

The term “shoot” means an organ of the Hoodia plant that grows above the soil surface. The shoot originates from the apical meristem plus one to several primordial leaves and also includes buds (small swelling) that will eventually develop into shoot.

The term “root” means that organ of the Hoodia plant that typically ties below the surface of the soil to anchor the plant. This is the vital organ that absorbs water and minerals which are essential for the plant growth and development.

“μM” means micromolar.

### Hoodia Plants

The invention is useful for an in vitro rooting of Hoodia plants. The Hoodia genus includes but is not Limited to Hoodia gordonii, Hoodia currorii, Hoodia juttae, Hoodia dregei, Hoodia parviflora, Hoodia pilifera, Hoodia alstonii, Hoodia flava, Hoodia officinalis, Hoodia mossamedensis, Hoodia triebneri, Hoodia pedicellata, Hoodia rushii. The invention is particularly valuable for Hoodia gordonii plants, as the actives derived from Hoodia gordonii plants have optimum safety and efficacy for weight management products. The demand for appetite suppressant actives derived from the Hoodia plants continues to grow.

### Explant

An explant (a piece) of the Hoodia plant for rooting is preferably obtained from micropropagated explants, which, in turn, are obtained via a multiplication step discussed in greater detail below. Alternatively, the explant may be obtained from a is field or from the greenhouse, in which case the explant is rooted directly, without the benefit of prior multiplication. Preferably, a shoot tip from a healthy, vigorous growing plant is obtained, preferably by cutting it off the mother plants. The length of the suitable explant piece is typically from 2 to 7 cm, preferably from 3 to 5 cm.

### Rooting

The rooting medium according to the invention includes basal salts, vitamins, two different auxins, gibberellin, and charcoal, in order to promote root formation and to avoid or minimise excessive callus growth at the cutting base. Callus formation generally slows down or in some cases prevent the rooting process. Roots formed with callus growth are not as vigorous as roots without callus i.e it takes a Longer period for the roots to be established in the greenhouse and they are more vulnerable to root rot. In the preferred embodiment of the process, callus formation is minimised or avoided.

According to the present invention, a mixture of at least two auxins is employed. The first auxin is indole-3-butyric acid (IBA) or its derivative indole-3-butyl-beta-alanine, or mixtures thereof. The second auxin is selected from the group consisting of α-naphthalene acetic acid (NAA), 2-naphthyoxyacetic acid (NOA), and mixtures thereof.

The growth regulator, gibberellin, is included. Suitable gibberellin is selected from the group consisting of gibberellic acid (GA₃, GA₄ and GA₇₎) and mixtures thereof and more preferably GA₃ which is a combination of GA₄ and GA₇. There are about 80 different gibberellins of which these three are most useful in promoting plant elongation.

The auxins and gibberellin are included according to the present invention typically in the following amounts:

| | Generally | Preferably | Most preferably |
|---|---|---|---|
| First auxin (e.g. | 10 to 50 μM | 25 to 36 μM | 29.50 μM |
| IBA) | | | |
| Second auxin (e.g. | 3 to 11 μM | 5 to 11 μM | 5.37 μM |
| NAA) | | | |
| Gibberellin | 1 to 6 μM | 3 to 6 μM | 2.89 μM |

Basal salts and vitamins are preferably incorporated into the rooting medium via a basal salt and vitamin mixture. Suitable mixtures include but are not limited to Murashige & Skoog (M&S) medium (Murashige and Skoog 1962), or a Gamborg B-5 medium containing the micro and macro salts and M&S vitamins (Gamborg et al. 1968).

| Component(mg/L) | M [symbol] S mixture | Gamborg B5 medium |
|---|---|---|
| Ammonium nitrate | 1650.0 | |
| Ammonium sulphate | | 134.0 |
| Boric acid | 6.2 | 3.0 |
| Calcium chloride anhydrous | 332.2 | 113.24 |
| Cobalt chloride • 6H₂O | 0.025 | 0.025 |
| Cupric sulfate • 5H₂O | 0.025 | 0.025 |
| Na₂-EDTA | 37.26 | 37.3 |
| Ferrous sulfate • 7H₂O | 27.8 | 27.85 |
| Magnesium sulfate | 180.7 | 17.099 |
| Manganese sulfate • H₂O | 16.9 | 10.0 |
| Molybdic acid(sodium salt) • | 0.25 | 0.25 |
| 2H₂O | | |
| Potassium iodide | 0.83 | 0.75 |
| Potassium nitrate | 1900.0 | 2500.00 |
| Potassium phosphate monobasic | 170.0 | |
| Sodium phosphate monobasic | | 130.5 |
| Zinc sulfate • 7H₂O | 8.6 | 2.0 |
| myo-Inositol | 100.0 | 100.0 |
| Nictotinic acid(free acid) | 0.5 | 1.0 |
| Pyridoxine • HCl | 0.5 | 1.0 |
| Thiamine • HCl | 0.5 | 10.0 |

The preferred basal salt mixture is M&S medium, at half strength salt concentration and vitamins at full strength concentration.

The rooting medium also comprises a carbohydrate source, e.g. starch or sugar. Carbohydrate source in the medium plays a major role in photosynthesis of the plants. Suitable sugars include but are not limited to sucrose, fructose, galactose, glucose, raffinose or maltose, more preferably sucrose and glucose and most preferably sucrose. The sugar is included typically in a concentration of from 10 to 40 g/l, preferably from 20 to 30 g/l and most preferably 30 g/l.

The rooting medium also contains charcoal. In addition to removing growth inhibitory substances and absorbing toxic substances which may be present in media ingredients as a result of autoclaving or exuded from cultured tissues, charcoal also helps in leaching excess cytokinin and auxin in the media, thus reducing callus growth and allowing plants to start producing their natural rooting hormones in vitro. Charcoal is included in the concentration of from 0.5 to 4 g/l, more preferably from 1 to 3 g/l, most preferably from 1 to 2 g/l.

Preferably, an antibiotic is included, e.g. cefotaxime.

The explants are placed onto rooting medium 1-2 cm deep in the medium in an erect position. The distance between the explants is the same as for the multiplication phase, although they may be placed closer (i.e. about 0.5-1 cm apart), as they are no longer producing new shoots.

The rooted plans are kept in the rooting medium under conditions of darkness at temperature of 26±2° C., typically for about 2-3 weeks, and later are placed under light until roots develop before going into the greenhouse for hardening.

### Multiplication

Preferably, the explants obtained from a field or greenhouse are first subjected to multiplication, to obtain a plurality of new shoots, and then rooted. The explants are obtained from the field or the greenhouse as described above, and then prepared for tissue culture multiplication medium as soon as possible, to avoid desiccation and build-up of exogenous contaminants (bacteria and fungus). The preparation steps include sterilization (optionally preceded by cleaning) and, optionally, de-spiking the explant. De-spiking involves carefully removing the spikes, 1 to 2 cm from the lower meristem e.g. with a surgical blade. It was found, as part of the present invention, that de-spiking of Hoodia explants results in a higher rate of introducing clean plants in the initiation medium because most of the explant tissues have a better contact with the sterilizing solution. The explants are preferably cleaned before sterilization, especially if explants are obtained from the field. The cleaning is best done under running tap water for a few minutes, typically from 2 to 10 minutes, preferably from 3 to 5 minutes, so as to remove the debris from the field. The explants are then placed in sterile containers and sterilized. Suitable sterilization is by treatment of explants with mercuric chloride (0.1% solution), typically for 2 to 10 minutes, preferably from 3 to 6 minutes, followed by treatment with sodium hypochlorite solution (30% solution) for 25-40 minutes, more preferably for 20 to 30 minutes in order to obtain clean plants which also survive sterilization and are able to produce new shoots. The sterilized explants are placed into a multiplication medium. Preferably, the explants are placed into initiation medium prior to the multiplication stage, in order to ascertain that the plants are clean and free of bacterial or fungal growth. The initiation medium is of the same composition as the multiplication medium and may further comprise an antibiotic. The explants may typically be kept in the initiation medium for two to three weeks.

Multiplication medium employs cytokinin and, optionally, an auxin, to promote the formation of at least one new shoot, preferably 3 to 7 shoots (with an average of at least from 3 to 5 shoots, more preferably an average of 3.5 to 4 shoots), while still avoiding or minimizing profuse callus formation at the base which interferes with shoot and root formation.

Suitable cytokinins are selected from adenine cytokinins and phenylurea cytokinins. Adenine cytokinin include but are not limited to kinetin, zeatin, and benzylaminopurine (BAP) (the tatter also known as benzyladenine). Phenylurea cytokinin or substituted phenylurea is selected from the group consisting of N,N′-diphenylurea and thidiazuron (TDZ), and 6-(dimethylallyamino)purine (2iP).

The preferred cytokinins are BAP, kinetin, zeatin and 2iP because they are commercially available. Most preferably, cytokinin is selected from BAP and kinetin and optimally is BAP.

Suitable auxins are broader than those described for the rooting medium above. Suitable auxins include natural and synthetic auxins. Natural auxins include but are not limited to indole-3-acetic acid (IAA) and its conjugates which include but are not limited to IAA alanine, IAA phenylalanine, IAA aspartic acid, IAA inositol and IAA acetylglycine. Synthetic auxins include but are not limited to 2,4-dichlorophenoxyacetic acid (2,4-D), α-naphthalene acetic acid (α-NAA), 2-methoxy-3,6-dichlorobenzoic acid (dicamba), 4-amino-3,5,6-trichloropicolinic acid (tordon or picloram), and 2,4,5-trichlorophenoxyacetic acid (2,4,5-T).

The preferred auxins are IAA and its conjugates, e.g. IAA alanine, IAA aspartic acid, IAA inositol and IAA phenylalanine which have high activity and are cheaper to use in tissue culture. Most preferably, auxin is selected from IAA alanine and IAA, and optimally is IAA.

The amounts of auxins and cytokinins in the multiplication medium are as follows:

| | Generally | Preferably | More Preferably | Optimally |
|---|---|---|---|---|
| Cytokinin | 2 to 60 μM | 4 μM to 45 μM | 9 μM to 25 μM | 17.74 μM |
| Auxin | 0 to 15 μM | 2 μM to 12 μM | 2 μM to 10 μM | 5.71 μM |

provided that when the auxin is present at a concentration above about 10 μM, the cytokinin concentration is below about 44 μM.

The same basal salts and vitamins as described above for rooting medium are suitable for the multiplication medium. Preferably M&S medium is employed at full strength salts and full strength vitamins, in order to minimize callus growth at the base of the shoots and some undesirable browning of the medium.

The multiplication medium preferably includes the same sugar and in the same concentration as described above for the rooting medium.

A preferred optional ingredient in the multiplication medium is gibberellin, in order to promote further shoot multiplication and also to promote shoot elongation Longer shoots are preferable, because they are easier to cut off for further propagation and at the later rooting stages root induction is better with longer shoots.

Gibberellin, preferably GA₃, is employed at a concentration ranging from 1 μM to 14 μM, preferably from 2 μM to 10 μM, more preferably from 3 μM to 8 μM, and most preferably at an optimum concentration of 5.77 μM. Gibberellin may be added to the multiplication medium just at the later cycles of multiplication, or may be present through all cycles of propagation.

Both the rooting and the multiplication medium preferably include a gelling agent. Suitable getting agents include but are not limited to gelrite, agarose, agar, starch, gellan gum and preferably are selected from gelrite and agar and most preferably gelrite, because it's a clear solidifying agent which makes it easier to see whether there is bacterial or fungal growth. Plants may also be grown on liquid medium without any getting agents.

Other suitable ingredients include coconut water which can serve as a vitamin supplement, and other basal salt mixes.

The explants are placed 1-1.5 cm deep into the sterile solidified multiplication medium in an erect position.

The spacing of explants, i.e. the distance between explants, plays an important role in successful multiplication. Hoodia shoots grow sideways and diagonally upwards and thus need sufficient room for successful growth. Too much space between the explants is undesirable, however, as it leads to over-spending of the multiplication containers and the medium, resulting in less commercially attractive process. It has been found that the optimum distance between the explants is from 0.5 cm to 3 cm, preferably from 1 to 2, and optimally 1.2-1.5 cm.

The explants are kept in the multiplication medium for a 3 to 4 week interval per cycle; at 26±2° C. in a medium where the pH is adjusted to 5.8 and the tubs are placed in a growth room on a bench with 16/8 h light dark photoperiod (39-90 μE/m²/s) provided by coot white fluorescent tubes.

To build up the stock, new shoots that originate from the mother plant are cut off and are transferred on a monthly basis onto fresh multiplication medium for further multiplication. For commercial micropropagation, the propagation cycle is repeated for 3 to 5 cycles after initiation, i.e. for the total of four to six months. Rooting can be done at any of these phases depending on the need. For commercial production, it is preferred to root the plants after the fifth cycle.

For commercial tissue culture micropropagation of Hoodia according to the present invention, from one single explant, it is possible to have approximately 600 to 800 plants after 5 to 6 months in the multiplication medium.

The explants with new shoots, or just the new shoots, are then transferred to a rooting medium for rooting, to obtain rooted plantlets, as described above. If it is the final stage of multiplication, one can cut off explants and put on the rooting medium and also transfer the mother plant for rooting. If at the early phase, say second phase, then its better to cut off new shoots for rooting and the mother plant for further multiplication.

It should be noted that various actives, including steroidal glycosides, may be extracted at any point during the inventive process, although their amount will be lower than in the mature plant, due to the size and maturity or age of the plant.

### Hardening

The hardening of the Hoodia plantlets is done, in order to acclimatize the rooted plantlet to in-vivo (external) conditions. It has been found, as part of the present invention that the micropropagated rooted Hoodia plantlets do not harden well under conditions that simulate wet dessert soil conditions, which may lead to vitrification (leaf or shoot having a glassy appearance) and fungal infestation. Rather, the plantlets are hardened best by using a gravel sort of “granite” mix, which allows for adequate drainage.

The plantlets are kept for hardening in a porous granitic fine mix and placed in a dry area with water being applied as a mist once in two days to avoid bacterial or fungal growth. This is done for three weeks until new true roots are formed before they can be transferred to the greenhouse where they receive minimal watering, twice a week for 3-6 weeks before going to the field.

Preferably, the plantlets are transferred to the field after gradual acclimatization. After the plants have established, which wilt normally take around 2 months, they should be placed for one hour under external conditions and the next day for two hours until when they become acclimatized before they can be transplanted to the field.

The following specific examples further illustrate the invention, but the invention is not limited thereto.

In all the multiplication and later rooting trials, the experiments were carried out in a randomised factorial design with at least four replicates per treatment. The student's t-test was used to determine significant difference between the phytohormone treatments. P values ≦0.05 was considered significant.

### EXAMPLE 1

This example investigated various concentrations basal salt medium, vitamins (those in M&S medium, always included at full strength), and various phytohormones on rooting. Each experiment was replicated four times and statistical analysis has been carried out as described above. The results that were obtained are summarised in Table 1.

TABLE 1

| | Basal | | IBA | NAA | GA₃ | Charcoal | |
|---|---|---|---|---|---|---|---|
| Number | Medium | Sucrose g/l | (μM) | (μM) | (μM) | g/l | Observations |
| 1. | M [symbol] S | 30 | 9.80 | 5.37 | 1.44 | 2 | Rooted plants with |
| | | | | | | | callus at shoot |
| | | | | | | | base. |
| 2. | M [symbol] S | 20 | 9.80 | 5.37 | 2.89 | 2 | Hard callus at base |
| | | | | | | | of shoot. Root |
| | | | | | | | developed but had |
| | | | | | | | to grow through |
| | | | | | | | the callus. 3 |
| | | | | | | | months to roots. |
| 3. | M [symbol] S | 20 | 9.80 | 04.46 | 2.89 | 0 | Prolific callus |
| | | | | | | | development with |
| | | | | | | | roots taking a long |
| | | | | | | | time to develop |
| | | | | | | | because of callus |
| 4. | M [symbol] S | 20 | 9.80 | 0 | 2.89 | 0 | No callus. Tissue |
| | | | | | | | swelled at base |
| | | | | | | | and rooting was |
| | | | | | | | poor. |
| 5. | ½ M [symbol] S | 30 | 4.90 | 5.37 | 0 | 0 | Slight tissue |
| | | | | | | | browning. |
| | | | | | | | Moderate callus |
| | | | | | | | growth. Roots |
| | | | | | | | developed slowly. |
| 6. | ½ M [symbol] S | 30 | 19.60 | 5.37 | 0 | 0 | Shoots brown. |
| | | | | | | | Roots developed |
| | | | | | | | slowly through |
| | | | | | | | callus. |
| 7. | ½ MS | 30 | 19.60 | 5.37 | 0 | 0 | Callus developed |
| | | | | | | | at the base, no |
| | | | | | | | roots formed. |
| 8. | ½ MS | 30 | 29.50 | 5.37 | 0 | 0 | Prolific callus |
| | | | | | | | production, no |
| | | | | | | | roots. |
| 9. | ½ MS | 30 | 9.80 | 5.37 | 2.89 | 2 | No callus. No |
| | | | | | | | visible roots. |
| 10. | ½ MS | 30 | 19.60 | 5.37 | 2.89 | 2 | No callus. No |
| | | | | | | | visible roots. |
| 11. | ½ MS | 30 | 29.50 | 5.37 | 2.89 | 2 | Roots produced in |
| | | | | | | | one month. No |
| | | | | | | | callus growth at |
| | | | | | | | the base. |

Notes:
IBA = indole butyric acid;
NAA = napthalene acetic acid;
GA₃ = gibberillic acid

Notes: IBA=indole butyric acid; NAA=napthalene acetic acid; GA₃=gibberillic acid The results in Table 1 clearly show the effect of various phytohormones and charcoal on rooting. Most of the media tested produced a dense callus growth at the cutting base, making it hard for roots to develop. However, at a higher IBA concentration of 29.50 μM, welt defined roots were produced a month after subculture without callus. By using this approach, 75% of the plantlets rooted within 4 weeks in culture. Charcoal reduced callus growth in the rooting medium. The addition of charcoal at 2 mg/l helped in teaching excess added auxin in the media. It allowed the plants to start producing their natural rooting hormones in-vitro. When experiment 11 was repeated with just IBA, or just NAA alone, no roots could be obtained.

While the present invention has been described herein with some specificity, and with reference to certain preferred embodiments thereof, those of ordinary skill in the art wilt recognize numerous variations, modifications and substitutions of that which has been described which can be made, and which are within the scope and spirit of the invention. It is intended that alt of these modifications and variations be within the scope of the present invention as described and claimed herein, and that the inventions be limited only by the scope of the claims which follow, and that such claims be interpreted as broadly as is reasonable.

What is claimed is:

1. A process of propagating Hoodia plants, the process comprising:
rooting the explant by placing the explant in contact with a rooting medium comprising:
(a) basal salts;
(b) vitamins;
(c) a carbohydrate source,
(d) a first auxin selected from the group consisting of indole-3-butyric acid, indole-3-butyl-beta-alanine, and mixtures thereof, at a concentration of from about 10 μM to about 50 μM,
(e) a second auxin selected from the group consisting of α-naphthalene acetic acid, 2-naphthyoxyacetic acid, and mixtures thereof, at a concentration of from about 3 μM to about 11 μM,
(f) giberellin at a concentration of from about 1 μM to about 6 μM;
(g) charcoal at a concentration of from about 0.5 g/L to about 4 g/L,
growing the Hoodia explant on the rooting medium, to obtain a rooted plant.

2. The process of claim 1, wherein the rooted plant is obtained within in the time span of from about 2 weeks to about 5 months.

3. The process of claim 1, wherein the rooted plant is obtained within the time span of from about 3 weeks to about 3 months.

4. The process of claim 1 wherein the first auxin is present at a concentration of from about 25 μM to about 36 μM.

5. The process of claim 1 wherein the second auxin is present at a concentration of from about 5 μM to about 11 μM.

6. The process of claim 1 wherein the carbohydrate source is sugar and it is present in an amount of from about 10 g/L to about 40 g/L.

7. The process of claim 1 wherein the giberrelin is present in an amount of from about 3 μM to about 6 μM.

8. The process of claim 1, wherein the rooting medium further comprises an antibiotic.

9. The process of claim 1 wherein the basal salts and vitamins are incorporated by a basal salt and vitamin mixture, wherein salts are at half strength and vitamins are at full strength.

10. The process of claim 1, wherein the rooting medium further comprises gelrite.

11. The process of claim 1, wherein the process further comprises placing a Hoodia explant in a multiplication medium prior to step (ii) to obtain an explant with new shoots, cutting off the new shoots and rooting the new shoots according to the process of claim 1.

12. The process of claim 11, wherein the multiplication medium comprises:
i. basal salt and vitamin mixture,
ii. carbohydrate source,
iii. a cytokinin at a concentration of from about 2 μM to about 60 μM,
iv, an auxin at a concentration of from about 0 μM to about 15 μM, provided that when the auxin is present at a concentration above about 10 μM, the cytokinin concentration is below about 44 μM; and

13. The process of claim 12 wherein the auxin is present in the multiplication medium at a concentration from about 2 μM to about 10 μM.

14. The process of claim 1 wherein the Hoodia plant is selected from Hoodia gordonii, Hoodia currorii, Hoodia juttae, Hoodia dregei, Hoodia parviflora, Hoodia pilifera, Hoodia alstonii, Hoodia flava, Hoodia officinalis, Hoodia mossamedensis, Hoodia triebneri, Hoodia pedicellata, Hoodia rushii.

15. The process of claim 1 wherein the Hoodia plant is Hoodia gordonii.

16. The process of claim 1 further comprising hardening the rooted plants.
